SEQUENCE LISTING (1 sequence)

SEQ ID NO: 1 - protein, 57 aa, Homo sapiens
TPDVSSALDKLKEFGNTLEDKARELISRIKQSELSAKMREWFSETFQKVKEKLKIDS

(19) United States
(12) Patent Application Publication
(10) Pub. No.: US 2010/0159492 A1
(43) Pub. Date: Jun. 24, 2010

(54) Method for the Diagnosis of Diseases by Determining Apolipoprotein C-1

(76) Inventor: Andreas Bergmann, Berlin (DE)

Correspondence Address: HESLIN ROTHENBERG FARLEY & MESITI PC, 5 COLUMBIA CIRCLE, ALBANY, NY, 12203, US

(21) Appl. No.: 10/572,522

(22) PCT Filed: Sep. 7, 2004
(86) PCT No.: PCT/EP04/09965

§ 371 Date: Dec. 18, 2007

(30) Foreign Application Priority Data
Sep. 22, 2003 (DE) 103 43 815.7

(51) Int. Cl. G01N 33/574; G01N 33/53; G01N 33/566; G01N 33/50

(52) U.S. Cl. 435/7.94; 436/86; 436/501; 436/64

(57) ABSTRACT

A method for the diagnosis, early detection, risk estimation and control of the course of diseases, wherein the amount of apolipoprotein C-I and/or derivatives thereof is determined in a serum or plasma sample from a human patient and it is possible to conclude as to the presence of a disease, particular a tumoral disease or sepsis, on the basis of a result deviating significantly from the value range determined for normal healthy persons. The invention also relates to the use of apolipoprotein C-I in the therapy and prevention of diseases wherein the amount of or binding ability of apolipoprotein C-I in the blood of sick persons is significantly different from that of normal persons.

The present invention relates to a method for the diagnosis, early detection, risk estimation and monitoring of the course of diseases, in which the protein apolipoprotein C-I is determined as a biomarker in serum or plasma samples from human patients, and the use of apolipoprotein C-I as a therapeutic active substance.

It should be noted at the outset that when the term “diagnostic method” is used generally in the following description, this term is as a rule intended for the sake of simplicity to represent methods for the diagnosis, early detection, risk estimation and monitoring of the course, including therapy-accompanying monitoring of the course, of diseases, unless it is evident from the specific context that only measurements with specific limited objectives are meant at the respective point.

Furthermore, it should be noted that, in the context of the present application, the term “diseases” is used for, as a rule, severe chronic and in particular acute diseases and is not intended to relate to disorders of fat metabolism, as may be regarded as risk factors for the development of atherosclerosis. In particular, cancer diseases and sepsis are to be singled out as “diseases”. However, the determination according to the invention can also be used in association with diagnostic methods which are intended to provide more detailed information about the state of a patient who is suffering from other diseases, in particular an acute heart disease, such as a cardiac infarction, angina pectoris and an arterial occlusive disease, or from diabetes, Graves' disease and Crohn's disease.

Proteins which form the lipoproteins together with lipids (triglycerides, cholesterol, phospholipids) are designated as “apolipoproteins”. An important function of the apolipoproteins is to permit the transport of the water-insoluble lipids in serum and plasma. On the basis of their migration behavior in the density gradient of an ultracentrifuge, lipoproteins are assigned to four different density classes: chylomicrons, VLDL (from: very low-density lipoproteins), LDL (from: low-density lipoproteins) and HDL (from: high-density lipoproteins). In addition, a group designated as lipoprotein (a) is distinguished. The lipid fraction of the lipoproteins is smaller the higher the density thereof. Lipoproteins of different density classes differ not only on the basis of the amount of lipid fraction present altogether but also with regard to the composition thereof. It is furthermore possible to assign typical apolipoprotein profiles to the respective density class.

Apolipoproteins are proteins of different length and amino acid composition, which are assigned to groups designated by A-H, a distinction being made in turn within the individual groups between different proteins, which are characterized by an attached number. Instead of the complete designation of, for example, apolipoprotein A-I, it has also been customary to speak only of Apo A-I. The primary structures (amino acid sequences) of the various apolipoproteins are known, and data or specific concepts regarding their three-dimensional structure also exist for a number of different apolipoproteins, in particular in association with lipids. More detailed information is to be found in the relevant scientific literature.

It is known that apolipoproteins can also be determined specifically in connection with the obtaining of measured data on the fat metabolism of a patient, in particular for early detection of a risk of atherosclerosis or for monitoring therapy in the treatment of a patient with lipid-lowering medicaments. Use is made of the circumstance that certain apolipoproteins occur exclusively or at least predominantly in lipoproteins of the individual lipoprotein density classes among the above-mentioned ones and determine the specificity thereof, so that, by determining the apolipoprotein fraction of lipoproteins, it is also possible to determine the proportion of the latter in a serum or plasma of a patient.

It is also known that lipoprotein and also apolipoprotein profiles differing from the normal case are found in individual patients owing to considerable effects which influence the formation of the apolipoproteins or the enzymes cleaving them or manifest themselves as receptor defects. It is furthermore known that disturbances of fat metabolism (secondary dyslipoproteinemias), which are of a temporary nature and disappear again after curing of the disease can also occur as a result of other diseases (cf. (20), pages 186-189; literature references in the form of numbers in brackets relate to the attached list of literature references). Where the changes in the fat metabolism also manifest themselves as changes in the apolipoprotein compositions, the scientific literature documents primarily those changes which relate to the apolipoproteins A-I and A-II and as a rule reflect abnormal forms and concentrations of the HDL. However, apolipoproteins do not play any significant practical role as biomarkers for the diagnosis of diseases which are also known to be accompanied by disturbances of fat metabolism.

The present application relates to the determination of apolipoprotein C-I for diagnostic purposes. The apolipoprotein C-I belongs to a group of apolipoproteins having comparatively low molecular weight, which are designated as apolipoproteins Apo C-I, Apo C-II and Apo C-III. The primary structures of these three proteins are known (cf. (1), (2) and, with regard to apolipoprotein C-I, in particular (3)). Relatively little is known about the function of the apolipoproteins C-I and C-III, and no documented knowledge is available concerning changes of the amounts or forms and association forms of apolipoprotein C-I in human serum or plasma as a function of certain pathological conditions. Publications according to (1) to (18) are representative of publications in the scientific literature which relate to apolipoproteins of group C or apolipoprotein C-I, in particular the work according to (11), providing a good overview in relation to what is known about the role of the apolipoproteins C in lipoprotein metabolism. The biological effects described for apolipoprotein C-I primarily include the inhibition of cholesterol ester transfer protein (CETP), the inhibition of the protein (LRP) associated with the LDL receptor (LDLR) and of LDLR itself and of the VLDL receptors (VLDLR), and the activation of lecithin cholesterol acyltransferase (LCAT).

No knowledge which would permit a determination of apolipoprotein C-I as a biomarker for the diagnosis of diseases, such as, for example, sepsis or cancer, or would suggest a determination of apolipoprotein C-I in said diagnostic contexts can be derived from the content of said publications.

The present invention is the result of systematic research work by the Applicant which is aimed at researching the changes in the protein composition of patients with sepsis and making the research results obtained usable specifically for the diagnosis of sepsis and inflammation.

A starting point of these researches was the finding that, in the case of sepsis, the prohormone procalcitonin is drastically increased and that the determination of procalictonin as a biomarker for sepsis is of great practical value for diagnosis and for therapy-accompanying monitoring (cf. for example EP 656 121 B1). Further discoveries relating to the change in the protein composition in the serum or plasma of sepsis patients, which were obtained by the Applicant starting from different approaches to research, are to be found, for example, in the published Patent Application EP 1 121 600 A2 and further published Patent Applications of a more recent date, such as WO02/085937, WO03/005035, WO03/002600, EP 1 355 158 A1, EP 1 318 406 A1, EP 1 318 407 A1 and EP 1 355 159 A1, and additional relevant still unpublished patent applications of the Applicant. Reference is made to the content of the said patent applications, in particular with regard to the explanations of the modern understanding of the term sepsis and the significance of the determination of biomarkers in the case of sepsis.

Since some of the investigations had shown that a number of biomarkers which were traditionally regarded only as typical cancer markers (cf. EP 1 318 402 A1, EP 1 318 403 A1, EP 1 318 404 A1 and EP1 318 405 A1) are significantly increased in the case of sepsis too, plasma and serum samples of patients who suffered from various malignant tumor diseases were increasingly taken into account alongside the investigation of samples of sepsis patients. In many of the cases investigated, it was found that individual biomarkers which are found at a raised level in the case of sepsis are also significantly raised in the case of cancer patients. However, numerous cases also revealed substantial differences which prevent sepsis diseases and malignant tumor diseases from being treated for the sake of simplification generally as being fundamentally similar.

The invention described in the present application is the result of a further research approach for determining proteins which are potentially suitable as biomarkers for diagnostic purposes because their measurable amounts in sepsis patients and possibly patients who are suffering from other severe diseases, in particular inflammatory diseases or cancer diseases, are significantly changed compared with healthy persons. For this purpose, HPLC profiles of serum or plasma fractions from sepsis patients were compared with those of apparently normal healthy persons, and significant changes in the HPLC chromatography profiles were subjected to a closer examination. These investigations gave the results which became the basis of the present invention, namely that significant changes in the HPLC elution profiles of serum or plasma samples of patients are detectable when these samples were separated beforehand over a column containing a chromatography material which is capable of interacting with hydrophobic or lipophilic constituents of the sample and selectively binding such constituents (“hydrophobic interaction chromatography”). If the constituents of the separated sample which are bound to the chromatography material are subsequently eluted in a suitable manner, a subfraction of the original serum or plasma sample is obtained, in which constituents, in particular those of a protein nature, which are capable of interacting with hydrophobic surfaces are selectively enriched. A variant of such a method as applied to lipoproteins is described in (19).

The separation of such fractions of plasma or sera of patients with sepsis and other diseases by means of high performance liquid chromatography (HPLC) led to the surprising discovery, on which the present invention is based, that a constituent which is to be found at a substantial level in sera and plasmas of normal healthy persons is greatly reduced or apparently completely absent in the sera of sepsis patients and certain other patients. Further investigations into the nature of this constituent, which are described in more detail below, showed that the significantly reduced constituent is apolipoprotein C-I. Subsequent further investigations, which are likewise described in more detail below, then led to the further surprising result that findings which, in an initial provisional evaluation, indicated a parallelism of the changes in sepsis and cancer patients are in reality to be clearly distinguished from one another and make it possible clearly to distinguish sepsis diseases from cancer diseases by measurement of apolipoprotein C-I in serum or plasma from human patients, which as such, constitutes a discovery which can be utilized for diagnostic purposes.

An object of the present invention is to find a novel protein biomarker suitable for diagnostic determinations and to make it usable for diagnostic purposes, the quantitative occurrence of which biomarker in human serum or plasma differs significantly from the values found in apparently normal healthy persons and the determination of which therefore constitutes a valuable diagnostic tool.

This object is achieved by a method as claimed in claim 1.

Preferred developments, in particular with regard to diagnostic applications in the case of sepsis patients, cancer patients and patients with other diseases are stated in the subclaims.

Where, in the present application or the claims, in addition to apolipoprotein C-I, “derivatives thereof” are also mentioned as species to be determined, these are to be understood as meaning in particular fragments and aggregates, in particular those which behave like the free protein apolipoprotein C-I in the respective chosen assay method. The “derivatives” may be, for example, apolipoprotein C-I molecules shortened by individual amino acids or amino acid sequences, or complete apolipoprotein C-I molecules sterically or conformationally modified—for example by aggregation.

The novel discoveries which permit the use of apolipoprotein C-I as a biomarker indicate that, in patients who are suffering from relevant diseases, the detectable amount of apolipoprotein C-I is greatly reduced compared with apparently normal healthy persons. From this it is possible to conclude that apolipoprotein C-I is formed in insufficient amounts, consumed and/or abnormally bound in the case of said diseases, which may be interpreted as a deficiency of apolipoprotein C-I in comparison with healthy persons.

According to a further aspect, the invention therefore also relates to the use of apolipoprotein C-I as a therapeutic agent for the treatment of disease-related apolipoprotein C-I deficiency conditions and for the preparation of medicaments for the treatment of diseases which, at the diagnostic level, manifest themselves through a significant reduction of the measurable amounts of apolipoprotein C-I.

These aspects of the invention form the subject matter of claim 10.

All facts and teachings to be protected which are reproduced in the introduction and in the claims to which reference is made are explained in more detail below with reference to experimental data and associated figures, the meaning of individual terms used in the present description being explained in addition. Express reference is therefore made to the following statements for the interpretation of the patent claims and for the more precise interpretation of the general statements made so far.

In the figures:

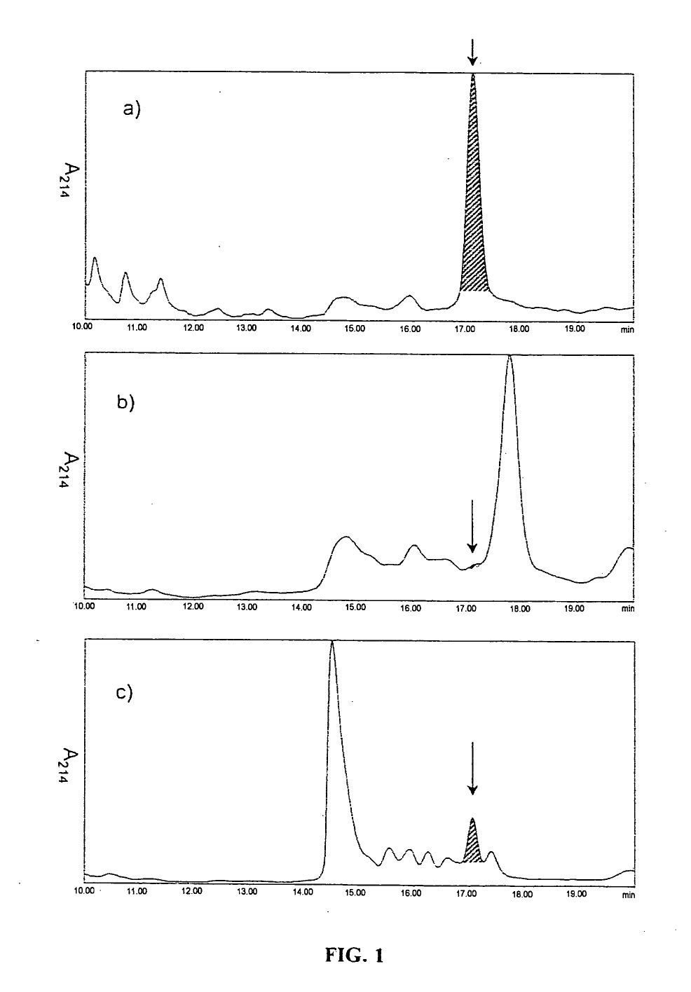

FIG. 1 shows the elution profiles of the reverse phase C18 HPLC of serum and plasma fraction which were elutable with acetic acid (50 mM) from octylsepharose chromatography columns which had been loaded with sera or plasma of (a) apparently normal healthy persons, (b) patients with various malignant tumor diseases and (c) sepsis patients, after intermediate washing of the loaded columns with phosphate-buffered saline solution (PBS).

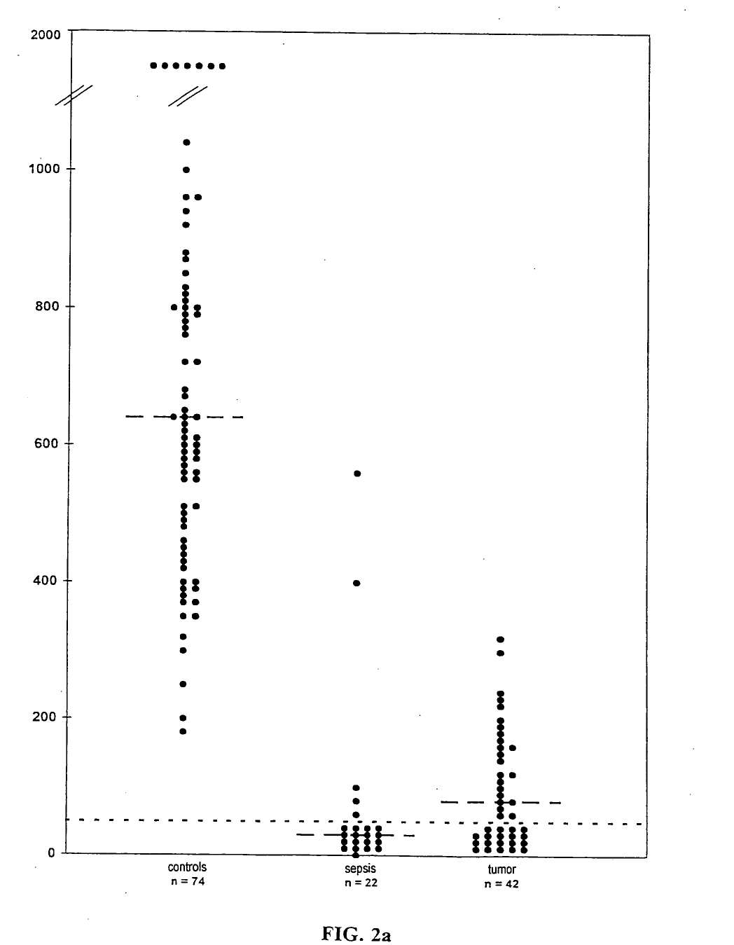

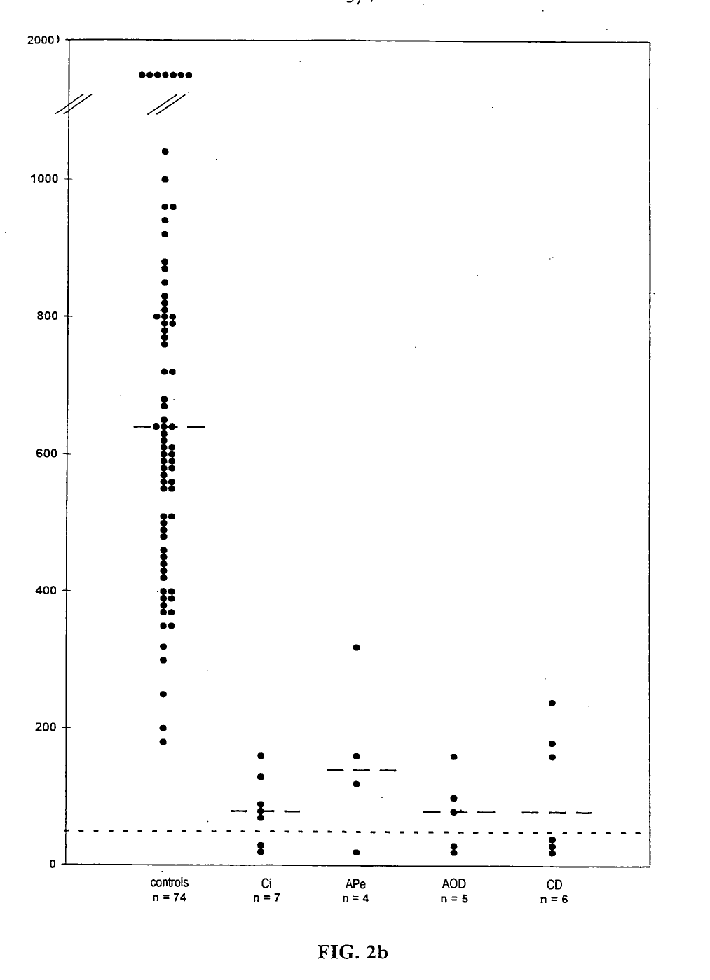

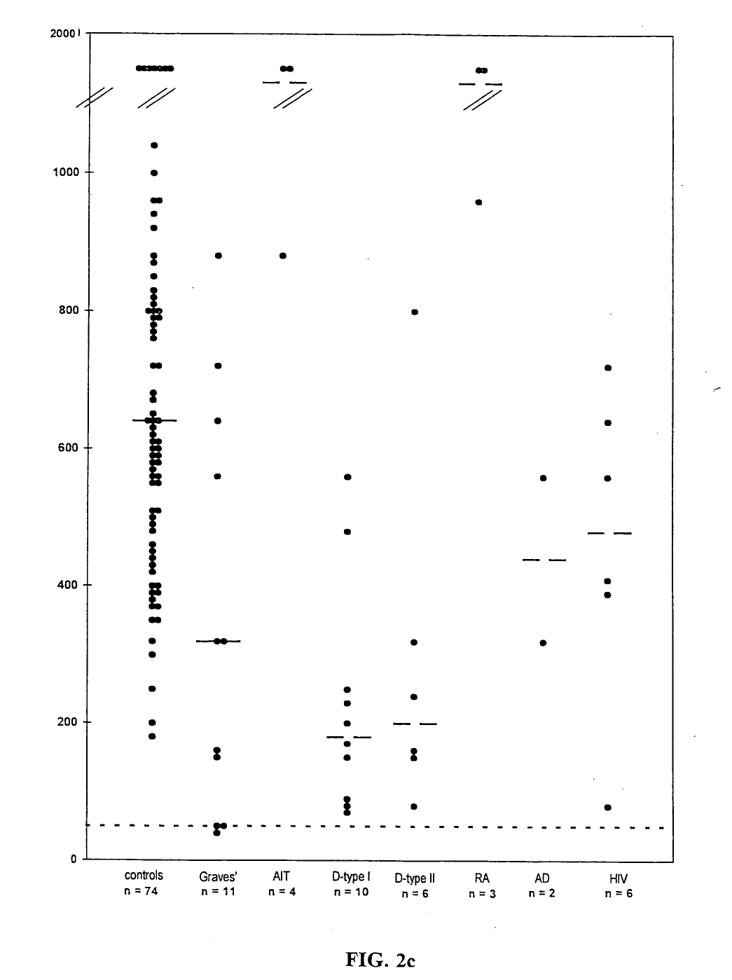

FIG. 2 shows relative amounts, obtained by peak integration of HPLC elution profiles of the type shown in FIG. 1, of elutable apolipoprotein C-I in samples of apparently normal healthy persons and patients who suffered from diseases of different types (sepsis, tumor, cardiac infarction, angina pectoris, arterial occlusive diseases, Crohn's disease, Graves' disease, autoimmune thyroiditis, diabetes I, diabetes II, rheumatoid arthritis, Alzheimer's disease, HIV positive sera, in each case independently clinically confirmed diagnoses).

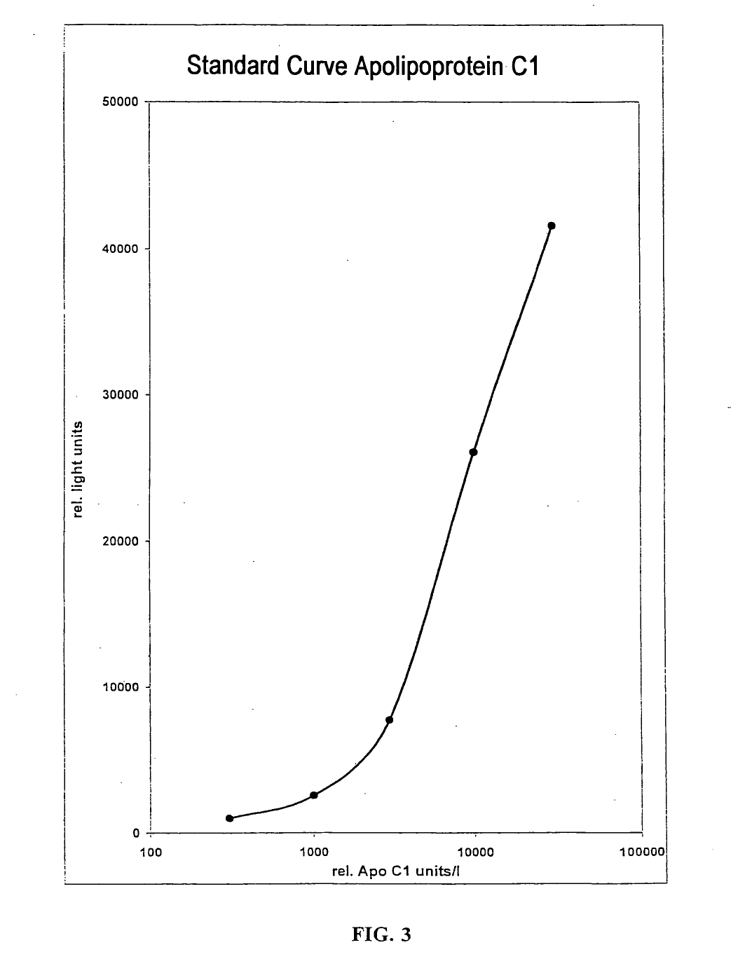

FIG. 3 shows the standard curve of a sandwich immunoassay operating with two affinity-purified polyclonal apolipoprotein C-I antibodies, as obtained using synthetic apolipoprotein C-I as standard.

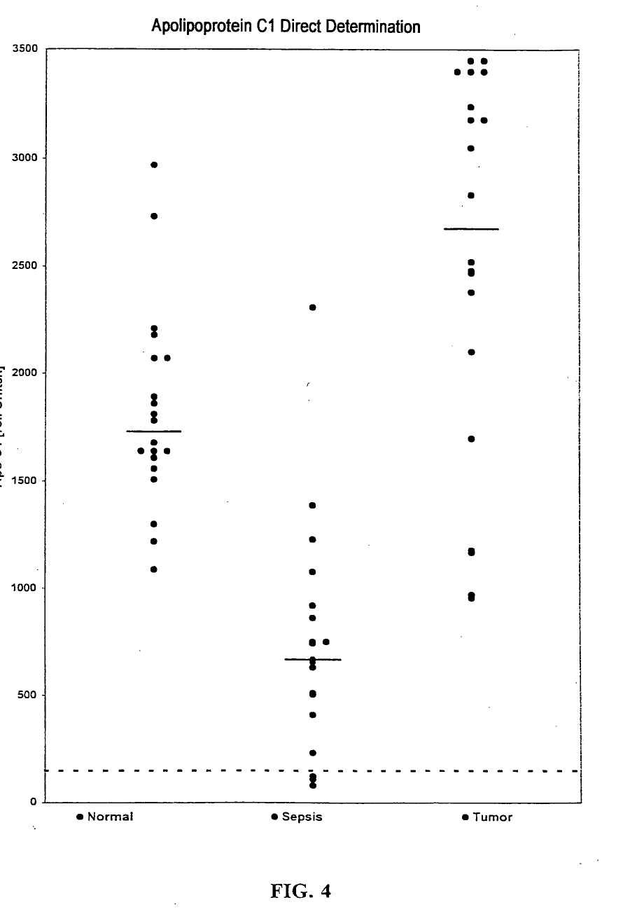

FIG. 4 shows the results of direct determination of apolipoprotein C-I in sera and plasma of apparently normal healthy persons, sepsis patients and tumor patients using an apolipoprotein C-I sandwich immunoassay, of which a typical calibration curve is shown in FIG. 3; and

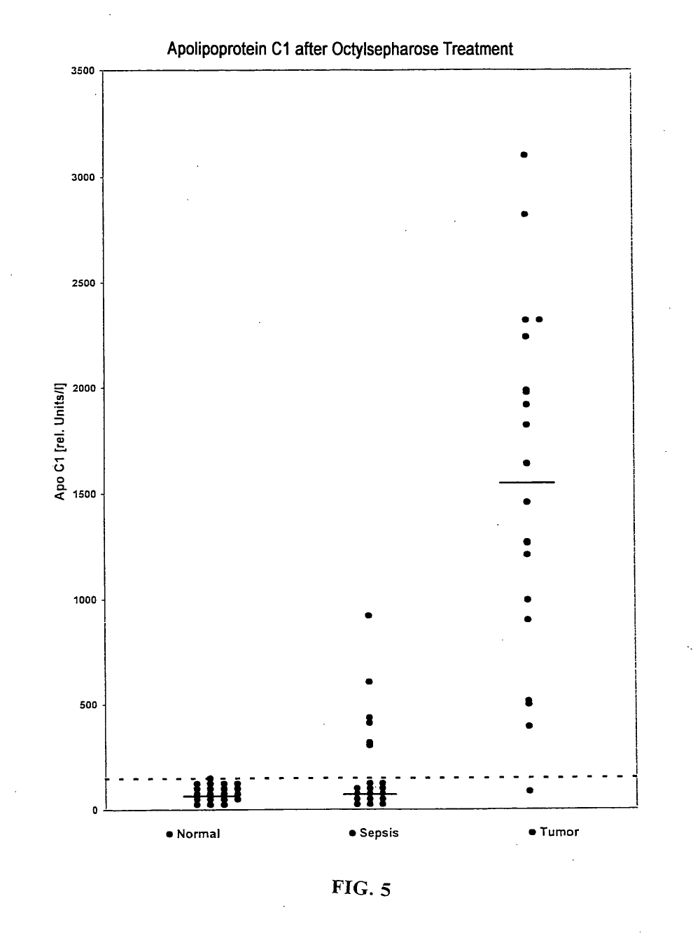

FIG. 5 shows the result of a repetition of the measurements, the results of which are shown in FIG. 4, after the respective samples had been treated with octylsepharose.

Description of the experiments carried out and of the results obtained

### A. Chromatographic Investigations

1. Obtaining Serum or Plasma Fractions which Contain the Constituents of Human Serum/Plasma which Bind to Hydrophobic Surfaces and can be Eluted Therefrom

Samples of in each case 0.5 ml of sera/plasma of apparently normal healthy persons and of patients who suffered from a number of independently clinically diagnosed diseases were mixed with 0.5 ml of octylsepharose chromatography material (source: Pharmacia; 0.25 ml of packed material, washed in PBS) in PBS and incubated for 10 min at room temperature with gentle shaking. Thereafter, the mixture was introduced into a polypropylene column (diameter 5 nm), and the unbound substances were separated from the substances bound to the octylsepharose material by washing with 20 ml of PBS.

The desorption of the substances (proteins) bound to the octylsepharose material was effected by elution with 50 mM acetic acid (pH 2.5).

2 Reverse phase C18 HPLC

The acetic acid eluate obtained from the octylsepharose columns was analyzed in each case directly by means of reverse phase C18 HPLC over a μ Bondapak C18 column (3.9×300 mm, Millipore, Waters).

Gradients of a mobile phase A (mixture of 95 parts by volume of water, 5 parts by volume of acetonitrile, 0.1 part by volume of trifluoroacetic acid) and a mobile phase B (10 parts by volume of water, 90 parts by volume of acetonitrile, 0.1 part by volume of trifluoroacetic acid) were used for the HPLC chromatography.

After application of the acetic acid eluate to the HPLC column, the analysis was effected in the following gradients of mobile phase A/mobile phase B:
t=0-5 min from 100/0 (A/B) to 65/35 (A/B)
t=5-20 min from 65/35 (A/B) to 40/60 (A/B)
t=20-22 min from 40/60 (A/B) to 0/100 (A/B)

The flow rate was 1 ml/min. The column outflow was measured continuously for absorption at 214 nm.

FIG. 1 shows three typical elution profiles for fractions of samples from apparently normal healthy patients (a), from patients with different tumor diseases (b) and from patients with sepsis (c).

It was surprisingly found that the HPLC elution profiles of said groups of persons differ substantially and systematically from one another. The band which is critical for the present application is that which is eluted at 17.05 min.

This band was quantified relatively by means of peak integration for all serum and plasma samples investigated, and the relative amounts of substance determined in this manner are shown graphically in FIG. 2 for a large number of control and patient samples. Specifically, the following samples were investigated:

42 samples from tumor patients (9× intestinal cancer, 7× bronchial carcinoma, 13× breast cancer, 8× ovarian carcinoma, 5× carcinoma of the pancreas), 22 samples from sepsis patients, 7 samples from cardiac infarction patients (Ci), 4 samples from patients with angina pectoris (APe), 5 samples from patients with arterial occlusive diseases (AOD), 6 samples from patients with Crohn's disease (CD), 11 samples from patients with Graves' disease, 4 samples from patients with autoimmune thryoiditis (AIT), 10 samples from patients with diabetes type I (D-type I), 6 samples from patients with diabetes type II (D-type II), 3 samples from patients with rheumatoid arthritis (RA), 2 samples from patients with Alzheimer's disease (AD) and 6 samples from HIV-positive patients. As is immediately evident from FIG. 2 substantially positive values for the substance which corresponds to the measured peak at 17.05 min are found for samples from normal healthy persons. In the samples from those suffering from diseases, very particularly clearly in the samples from sepsis patients, tumor patients, patients with manifest/acute cardiac diseases (Ci, APe, AOD) and patients with Crohn's disease, highly significantly reduced proportions of the same substance are measured. For patients with diabetes type I and diabetes type II too, significantly reduced values, although not reduced to the same extent, are found compared with healthy persons. For patients with Graves' disease, autoimmune thyroiditis, rheumatoid arthritis, Alzheimer's disease and HIV-positive patients the corresponding concentration deviations are smaller or statistically insignificant.

The mean values of the relative amounts found, which are obtained for the individual sample groups are summarized in the table below:

| | Mean value (relative |
|---|---|
| Sample type | absorption units) |
| Controls | 640 |
| Sepsis | 30 |
| Tumor | 80 |
| Cardiac infarction | 80 |
| Angina pectoris | 140 |
| Arterial occlusive diseases | 80 |
| Crohn's disease | 80 |
| Grave's disease | 320 |
| Autoimmune thyroiditis | 1130 |
| Type I diabetes | 180 |
| Type II diabetes | 200 |
| Rheumatoid arthritis | 1130 |
| Alzheimer's disease | 440 |
| HIV positive | 480 |

3. Identification of the Band Used for Sample Characterization (Band Eluting at 17.5 min)

For identification, the eluted substance corresponding to this band was collected, dried (speed vac) and subjected to peptide analysis. In a sequence analysis of the N-terminus the sequence TPDVS was determined. The molecular weight determined by means of the mass spectrum (ESI MS) is 6630±15 D. These results correlate unambiguously with the known data for human apolipoprotein C-I (cf. for example (3)), which has the peptide sequence according to SEQ ID No. 1 (cf. also SWISS PROT Accession No. 3660227) and has a theoretical molecular weight of 6627 D.

After trypsin digestion of the substance and subsequent mass spectroscopy (ESI MS; “trypsin fingerprinting”) the assignment made was completely confirmed.

The apolipoprotein C-I which can be determined by the chromatographic procedure described is also referred to as “free apolipoprotein C-I” in the context of the present application. It has the properties of binding from a serum or plasma sample (in PBS dilution) to hydrophobic molecular structures, for example the octyl radical of a hydrophobic octylsepharose chromatography material, from which it can be eluted under typical conditions for the elution of proteins, in particular with dilute acetic acid. However, the use of the term “free apolipoprotein C-I” does not necessarily mean that the material which can be separated off by hydrophobic interaction chromatography has to be present in completely free form in the original samples. Associates or aggregates, also with lipids which are broken up under the experimental conditions on contact with octylsepharose with binding of the apolipoprotein C-I to the chromatography material, are also to be regarded as “free apolipoprotein C-I” in the context of the use of this term in the present application.

The “hydrophobic molecular structures” can thus also be parts of the surface of a hydrophobic chromatography material. However, they may also be individual molecules having hydrophobic structural regions via which binding of such a molecule to apolipoprotein C-I can take place, for example molecules having hydrophobic structures which can be marked and, for example, can serve in homogeneous assays for marking apolipoprotein C-I in a sample fluid. The ability to bind to “hydrophobic molecular structures” is present when the regions of the apolipoprotein C-I in the sample which are available for such binding are not blocked by other substances, for example the lipids in lipoproteins. Even when, for example, it is stated that those fractions of apolipoprotein C-I are determined which bind to octylsepharose, this does not mean that it is actually necessary to make use of binding to octylsepharose in the determination. Rather, such a statement is to be understood as a characterization of the substance to be determined, even if it is determined by completely different methods. For example, the corresponding apolipoprotein C-I may be the same as that which is also determined, as described below, by means of an immunoassay.

### B. Direct Immunodiagnostic Determination of Apolipoprotein C-I in Sera/Plasmas

### 1. Apolipoprotein C-I Immunoassay

For the direct immunodiagnostic determination of apolipoprotein C-I in serum, an immunoassay of the sandwich type was set up from the components described below:

a) Coated tubes: polystyrene tubes (Greiner) were coated with a commercially available polyclonal affinity-purified antibody against apolipoprotein C-I (source: Acris Antibody, Bad Neuheim, Germany). According to the manufacturer's information, the antibody had been obtained by immunization of rabbits with human Apo C-I and had been purified over a Sepharose affinity column with human apolipoprotein C-I. For coating, 0.2 μg of antibody in 300 μl of PBS was bound to polystyrene tubes (Greiner, Germany) which had been coated with sheep anti-rabbit IgG antibodies (Sigma). The binding was complete after 18 hours at room temperature. The tubes were then washed twice with 3 ml portions of 0.5% bovine serum albumin (BSA) in PBS. After they had been dried in vacuo, the tubes were used as a solid phase for the apolipoprotein C-I immunoassay.
b) Acridinium ester-marked antibody: 100 μg of another antibody against human apolipoprotein C-I (from rabbits; source: Academie Bio-Medical Company, Texas, USA) in 100 μl of PBS were reacted with 10 μg of acridinium NHS ester (in 10 μl of acetonitrile). After incubation for 10 minutes at room temperature, the marked antibody was purified with separation of unconverted constituents of the reaction mixture by HPLC over SW 300 (Waters). For its use in the immunoassay, the marked antibody in PBS with 0.5% of BSA and 1 mg/ml of rabbit IgG was adjusted to about 1 million RLU/300 il (RLU=relative light units) for saturation of the tube walls.

### 2. Carrying Out the Immunodiagnostic Determination of Apolipoprotein C-I in Serum or Plasma Samples

Serum or plasma samples were diluted 1:10 000 with PBS, 0.5% BSA. In each case 300 μl thereof were pipetted into the abovementioned tubes coated with the immobilized antibody and were then incubated for 4 hours at room temperature with shaking (300 rpm on a Heidolph rotary shaker). The tube content was then washed out with PBS (filling and decanting 4 times with 1 ml of PBS in each case), and apolipoprotein C-I bound to the tube wall was reacted in the course of 20 hours at room temperature and 300 rpm with 300 μl per tube of acridinium ester-marked anti-apolipoprotein C-I antibody. Thereafter, unbound marked antibody was removed by washing 5 times with 1 ml portions of PBS, and the remaining chemiluminescence was measured in a known manner in a Berthold 952 T luminometer.

A synthetic apolipoprotein C-I was used for calibrating the assay. A typical standard curve as obtained for the above assay is shown in FIG. 3.

### 3. Results

With the aid of the described immunoassay of the sandwich type, controlled sera of apparently normal healthy persons, as were also used in the chromatographic investigations, and sepsis and tumor samples were measured. The measurements of the samples from sepsis patients substantially confirm the results of the HPLC investigation, in that significantly reduced measured values were obtained for apolipoprotein C-I compared with normal persons. In the measurement of samples from tumor patients, for which reduced concentrations were likewise determined in the chromatographic investigations, however, there was a tendency for the measured values obtained to be increased. The results are shown graphically in FIG. 4.

For checking the question as to whether the different results for tumor patients in the chromatographic determination on the one hand and the immunodiagnostic determinations on the other hand were due to the preceding selection step by means of hydrophobic interaction chromatography, the samples measured in the immunoassay were treated with octylsepharose before a further determination for the binding of “free” fractions of apolipoprotein C-I.

4. Treatment of the Samples with Octylsepharose

150 μl of the same serum or plasma samples which had been measured as described in the immunoassay were mixed with 200 μl of octylsepharose (Pharmacia, 50 μl of a gel in PBS) and incubated for one hour at room temperature with gentle shaking, after which the octylsepharose with apolipoprotein C-I fractions bound thereto were separated off by centrifuging (15 min at 2000 G). The supernatant obtained was diluted 1:5000 in PBS/0.5% BSA and then measured, as described above, in the immunoassay.

The results obtained are shown in FIG. 5. It is evident that, as a result of the treatment with octylsepharose, the total of “free” apolipoprotein C-I which is detectable in the measurement by the described immunoassay of the sandwich type had been bound to the octylsepharose so that apolipoprotein C-I was no longer detectable in the supernatant. Similar observations were made for the treated sepsis samples, in that the major part of the amounts of determinable apolipoprotein C-I which were already greatly reduced in comparison with the normal samples was removed from the sample by the treatment with the octylsepharose.

Surprisingly, the apolipoprotein C-I determinable in an immunoassay of the type described was, however, not removed by the treatment of octylsepharose from the samples of tumor patients.

### C. Discussion

The findings described show that at least a major part of the apolipoprotein C-I in sera or plasmas of tumor patients is present in a form in which it has lost its capability of binding to hydrophobic surfaces but can still be determined as immunoreactivity in an immunoassay. A possible explanation of these observations would be that, in the case of the apolipoprotein C-I occurring in sera of tumor patients, the molecular regions available for hydrophobic interactions are saturated or blocked, in particular by binding partners which cannot be displaced by the octylsepharose. The nature of these binding partners is currently unknown. However, it cannot be ruled out that they are tumor-specific substances which, as such or on the basis of the deactivation of hydrophobic binding partners, such as, for example, of apolipoprotein C-I, play an important role for tumor development, for example by having an adverse effect on the natural immune response or by actively promoting tumor growth. Although no significant reduction of the apolipoprotein C-I fraction is detectable in the samples investigated by the immunodiagnostic method, the parallel chromatographic investigations show that this is not “free” in the abovementioned sense but is present in another form with reduced capability of binding to hydrophobic binding partners. “Free” apolipoprotein C-I, i.e. apolipoprotein C-I binding to hydrophobic binding partners, is on the other hand reduced in sera of tumor patients as in the case of other diseases such as, for example, sepsis.

On the basis of the findings described, it may be presumed that it is advantageous for tumor patients to compensate their disturbed balance in the sense of a reduced proportion of “free” lipoprotein C-I by external supply of apolipoprotein C-I, and hence to have a positive influence on the pathological process, for example by saturating the binding partners typical for tumors by external apolipoprotein C-I and providing additional free apolipoprotein C-I.

A similar assumption, namely that an external supply of apolipoprotein C-I for compensating a disturbed level of “free” apolipoprotein C-I is advantageous, also applies to the other diseases, for example to the case of sepsis. If the production of apolipoprotein C-I is disturbed in the case of sepsis, an external supply can compensate a deficiency caused thereby. If the reduction of “free” apolipoprotein C-I in the serum/plasma of a patient in the case of sepsis is attributable to the removal of apolipoprotein C-I from the circulation by binding to pathological tissue structures or possibly insoluble bacterial structures, an external supply of apolipoprotein C-I can compensate the resulting deficiency or effect saturation of the binding sites in a pathological tissue or on the bacterial structures. By monitoring the concentration of free apolipoprotein increased by external supply or apolipoprotein determinable as immunoreactivity in blood samples of the patient treated, the amount required in each case can be monitored in a simple manner.

The present invention thus also permits a novel therapeutic use of apolipoprotein C-I or of suitable derivatives, or compounds simulating its physiological behavior, for the treatment of cancer, sepsis, and other diseases which are associated with a reduction of the detectable free apolipoprotein C-I compared with healthy persons.

### LIST OF REFERENCES

1. Erika Polz et al., Human Apolipoprotein C-I: Concentration in Blood Serum and Lipoproteins, Biochemical Medicine 24, 229-237 (1980)
2. Kane, J. P., in “Lipid Metabolism in Mammals” (F. Snyder, Ed.), Vol. 1, S. 209, Plenum, New York, 1977
3. Shulman, RS et al., The complete amino acid sequence of C-I (apoLP-Ser), an apolipoprotein from human very low density lipoproteins, J Biol Chem. 250, 182-190 (1975)
4. Xiao-Ren Pan et al., Abnormal Composition of Apolipoproteins C-I, C-II, and C-II in Plasma and Very-Low-Density Lipoproteins of Non-Insulin-Dependent Diabetic Chinese, Clin. Chem. 32, No. 10, 1914-1920 (1986)
5. Jutta Poensgen, Apolipoprotein C-I inhibits the hydrolysis by phospholipase A₂ of phospholipids in liposomes and cell membranes, Biochim. Biophys. Acta, 1042 (1990), 188-192
6. Ming Liu et al., Activation of plasma lysolecithin acyl-transferase reaction by apolipoproteins A-I, C-I and E, Biochim. Biophys. Acta, 1168 (1993), 144-152
7. Nina D. Bren et al., Quantification of Human Plasma Apo-lipoproteins C-I, C-II, and C-III by Radioimmunoassays, Mayo Clin Proc, July 1993, Vol. 68, 657-664
8. Rampratap S. Kushwaha et al., Characterization of cholesteryl ester transfer protein inhibitor from plasma of baboons (Papio sp.), J. Lipid Res. 1993, 34:1285-1297
9. N. Savion et al., Role of apolipoproteins A-I, A-II and C-I in cholesterol efflux froth endothelial and smooth muscle cells, Eur Heart J (1993) 14, 930-935
10. Janine H. van Ree et al., Inactivation of Apoe and Apoc1 by two consecutive rounds of gene targeting: effects on mRNA expression levels of gene cluster members, Human Molecular Genetics, 1995, Vol. 4, No. 8, 1403-1409
11. Miek C. Jong et al., Role of ApoCs in Lipoprotein Metabolism—Functional differences Between ApoC1, ApoC2, and ApoC3, Arterioscler Thromb Vasc Biol. 1999; 19:472-484
12. Thomas Gautier et al., Human Apolipoprotein C-I Accounts for the Ability of Plasma High Density Liproproteins to Inhibit the Cholesteryl Ester Transfer Protein Activity, J. Biol. Chem. 275, No. 48, 37504-37509, (2000)
13. Jong M. C. et al., Insights into Apolipoprotein C Metabolism from Transgenic and Gene-Targeted Mice, Int. J. Tissue React. XXII (2/3) 59-66 (2000)
14. Neil S. Shachter, Apolipoproteins C-I and C-III as important modulators of lipoprotein metabolism, Current Opinion in Lipidology 2001, 12:297-304
15. Benjamin W. Atcliffe et al., The interaction of human apolipoprotein C-I with sub-micellar phospholipid, Eur. J. Biochem. 268, 2838-2846 (2001)
16. Thomas Gautier et al., Apolipoprotein CI Deficiency Markedly Augments Plasma Lipoprotein Changes Mediated by Human Cholesteryl Ester Transfer Protein (CETP) in CETP Transgenic/ApoCI-knocked Out Mice, J. Biol. Chem. 277, No. 35, 31354-31363 (2002)
17. Puiying A. Mak et al., Regulated Expression of the Apoli-poprotein E/C-I/C-IV/C-II Gene Cluster in Murine and Human Macrophages, J. Biol. Chem. 277, No. 35, 31900-31908 (2002)
18. Johan Björkegren et al., Postprandial Enrichment of Remnant Lipoproteins With ApoC-I in Healthy Normolipidemic Men With Early Asymptomatic Atherosclerosis, Arterioscler Thromb Vasc Biol. 2002; 22:1470-1474
19. John G. Raynes et al., Purification of Serum Amyloid A and Other High Density Apolipoproteins by Hydrophobic Interaction Chromatography, Analytical Biochemistry 173, 116-124 (1988)
20. Lothar Thomas (Editor): Labor and Diagnose, 5. erweiterte Auflage, Frankfurt 200, Kapitel 4 Fettstoffwechsel, S. 177-190
21. Cesare R. Sitori, Evaluation of Lipoproteins/Apolipoproteins as Therapeutic Agents for the Treatment of Vascular and Nonvascular Disease, Am J Cardiol., Vol 81, 36F-39F (1998)

1. A method for the diagnosis, early detection, risk estimation and monitoring of the course of diseases, wherein the content of apolipoprotein C-I and/or of derivatives thereof is determined in a serum of plasma sample from a human patient and the presence of a disease is concluded on the basis of a result of the determination which differs significantly from the value range determined for normal healthy persons.

2. The method as claimed in claim 1, wherein that fraction of the total apolipoprotein C-I present in a sample which has the ability to bind to hydrophobic molecular structures (“free apolipoprotein C-I”), is determined.

3. The method as claimed in claim 1, wherein that fraction of the total apolipoprotein C-I present in a sample which is detectable by direct determination in the sample using an immunoassay of the sandwich type (“apolipoprotein C-I”) is determined.

4. The method of claim 1, wherein said method is carried out for the diagnosis, early detection, risk estimation and monitoring of the course of diseases which are selected from the group consisting of cancer diseases, sepsis, acute cardiac diseases, diabetes, Graves' disease and Crohn's disease.

5. The method of claim 1, wherein said method is carried out for the diagnosis, early detection, risk estimation and monitoring of the course of sepsis and correlates a proportion of apolipoprotein C-I which is significantly reduced compared with normal healthy persons and binds to hydrophobic molecular structures (“free apolipoprotein C-I”), and/or a reduced apolipoprotein C-I immunoreactivity in a serum or plasma sample of the patient with a septic condition.

6. The method of claim 1, wherein said method is carried out for the diagnosis, early detection, risk estimation and monitoring of the cause of a cancer disease and correlates a proportion of apolipoprotein C-I which is significantly reduced compared with normal healthy persons and binds to hydrophobic molecular structures (“free apolipoprotein C-I”) and an increased apolipoprotein C-I immunoreactivity in a serum or plasma sample of the patient with a cancer disease.

7. The method as claimed in claim 6, wherein, when an increased lipoprotein C-I immunoreactivity is found in a serum or plasma sample of a patient the measured value is checked by an additional control determination in which a check is carried out to determine whether the value for the determinable immunoreactivity in the sample is significantly changed by treatment of the sample with an adsorbent with hydrophobic surfaces, and wherein the presence of a cancer disease is concluded with high probability when the value does not deviate or does not deviate significantly from the original measured value.

8. The method of claim 1, wherein apolipoprotein C-I which binds to hydrophobic molecular structures (“free apolipoprotein C-I”) is determined by subjecting a serum or plasma sample to hydrophobic interaction chromatography, in which apolipoprotein C-I is bound to the chromatography material, and then determining apolipoprotein C-I in the eluted protein fraction.

9. The method as claimed in claim 8, wherein octylsepharose is used as chromatography material, unbound constituents of the sample are removed by washing the chromatography material, the bound proteins are eluted with a dilute acid, in particular acetic acid, and the amount of apolipoprotein C-I in the eluate is then determined by means of HPLC and/or immunodiagnostically.

10. (canceled)
